(19) United States
(12) Patent Application Publication
(10) Pub. No.: US 2019/0320757 A1
(43) Pub. Date: Oct. 24, 2019

(54) APPARATUS FOR ORTHOPEDIC USE

(71) Applicant: Thomas Pichler, Miami, FL (US)

(72) Inventor: Thomas Pichler, Miami, FL (US)

(21) Appl. No.: 16/393,917

(22) Filed: Apr. 24, 2019

Related U.S. Application Data
(60) Provisional application No. 62/662,166, filed on Apr. 24, 2018.

(51) Int. Cl. A43B 7/14; A61F 5/14; A43B 17/02

(52) U.S. Cl. CPC A43B 7/144; A61F 5/14; A43B 7/149; A61F 5/0127; A43B 7/142; A43B 17/026; A43B 7/141; A43B 7/1465

(57) ABSTRACT

The new apparatus is an orthopedic foot bed created using multiple materials to provide maximum comfort for an individual. The apparatus is composed of a flexible material such as, for example, memory foam on top of another material such as an impact absorbing material and an arch-support foot bed made of, for example, antimicrobial energy returning foam, with the entire apparatus covered in a stretchy suede textile in one embodiment.

### BACKGROUND OF THE SYSTEM

This patent application claims priority to U.S. Provisional Patent Application No. 62/662,166 filed on Apr. 24, 2018, which is incorporated by reference herein, in its entirety.

Orthopedic foot pads are used to reduce pain in a person's feet, legs and back and provide support. Orthopedic foot pads are commonly made from a single type of material such as memory foam or a gel material.

### SUMMARY

The new apparatus is an orthopedic foot bed created using multiple materials to provide maximum comfort for an individual. The apparatus is composed of a flexible material such as, for example, memory foam on top of another material such as an impact absorbing material and an arch-support foot bed made of, for example, antimicrobial energy returning foam, with the entire apparatus covered in a stretchy suede textile in one embodiment.

### BRIEF DESCRIPTION OF THE DRAWINGS

FIG. 1 depicts a cross section of an embodiment of the foot bed.

FIG. 2 depicts an angled view of an embodiment of the foot bed.

FIG. 3 depicts a cross section of another embodiment of the foot bed.

FIG. 4 depicts a cross section of an embodiment of the foot bed in an embodiment of a shoe.

### DETAILED DESCRIPTION OF THE APPARATUS

The apparatus is an orthopaedic foot bed composed of multiple materials to achieve maximum comfort for an individual.

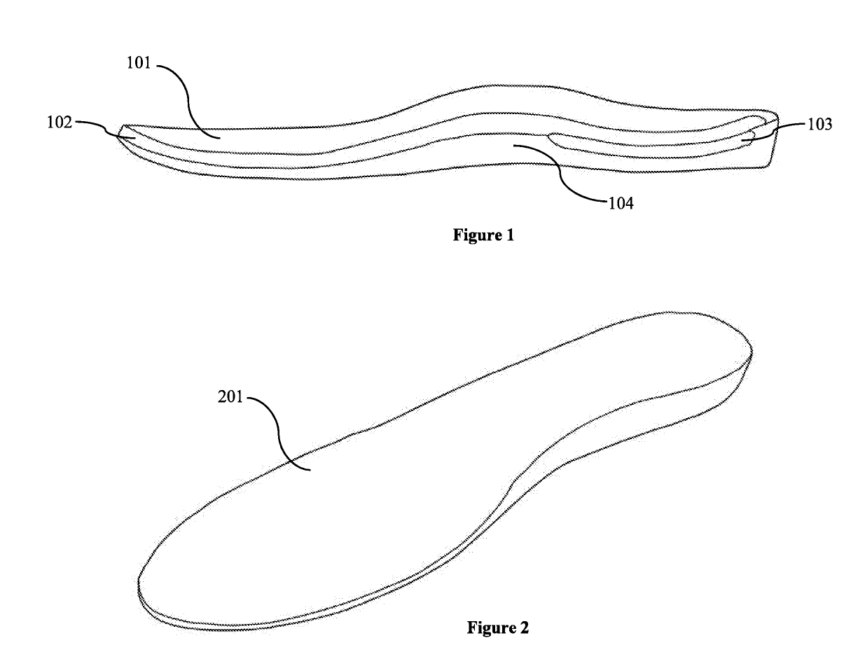

One embodiment of the apparatus can be seen in FIG. 1. In this embodiment the apparatus is composed of an arch-support foot bed 104 made of a light-weight antimicrobial energy returning foam to support the foot and allow it to breathe. A heel pad 103 made of an impact absorbing material with the following properties: a density of 16-28 lbs/ft³, and a compression set percentage of under 10%, for example, PORON® XRD® Extreme Impact Protection, is placed inside of the heel of the arch-support foot bed 104 to absorb the impacts that result from walking. A layer of memory foam 102 is placed on top of the arch-support foot bed 104 to provide support and comfort to an individual's foot. The entire apparatus is then enveloped in soft stretchy suede textile 101, which provides a stylish appearance, a soft feel, and allows the entire apparatus to be removed from a shoe in one piece. As seen in FIG. 2, the soft stretchy suede textile covering 201 covers the entire apparatus, covering the memory foam 102, heel pad 103, and arch support foot bed 104, preventing them from being seen when the apparatus is taken out of the shoe.

In one embodiment, the arch-support foot bed extends from the toe to the heel of the shoe. Similarly, the memory foam layer 102 extends substantially the length of the arch-support foot bed 104.

In one embodiment, the foot bed is placed in the embodiment of a shoe as seen in FIG. 4. The footbed 402 is placed inside of shoe 401. Forefoot outsole 403 and heel outsole 404 contain forefoot outsole pad 405 and heel outsole pad 406. Forefoot outsole pad 405 and heel outsole pad 406 are both made of a resilient shock absorbing material with the following properties: a density of 13-19 lbs/ft³, a compression set percentage of under 10%, a compression force deflection of of 70-170 kPa, and a vertical rebound greater than 35, for example, PORON® Vive® Energy Activated Cushioning to help push the foot into the next step while walking.

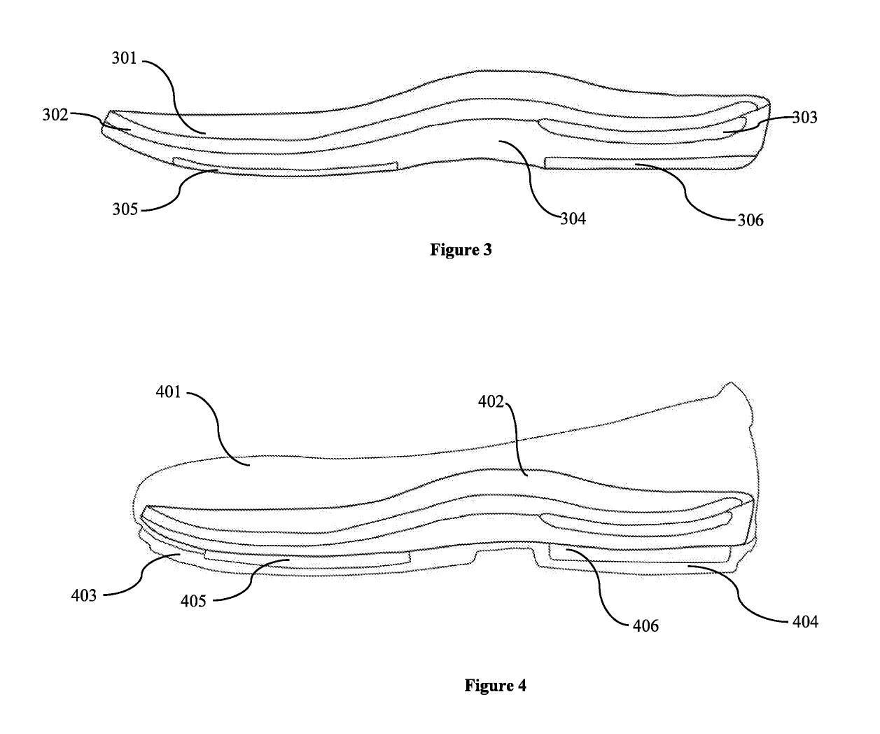

Another embodiment of the apparatus can be seen in FIG. 3. In addition to soft stretchy suede textile covering 301, memory foam 302, heel pad 303, and arch support foot bed 304, forefoot pad 305 and lower heel pad 306 are located on the bottom of the apparatus below arch support foot bed 304. Forefoot pad 305 and lower heel pad 306 are both made of a resilient shock absorbing material with the following properties: a density of 13-19 lbs/ft³, a compression set percentage of under 10%, a compression force deflection of of 70-170 kPa, and a vertical rebound greater than 35, for example, PORON® Vive® Energy Activated Cushioning to help push the foot into the next step while walking.

Thus an apparatus for an orthopaedic foot bed has been described.

What is claimed is:

1. An orthopedic appliance comprising:
an arch-support foot bed made of anti-microbial, energy returning foam;
a heel pad made of an impact absorbing material inlaid in said arch-support foot bed;
a memory foam foot pad resting on top of said arch-support foot bed and heel pad;
a suede covering that covers the top of said arch-support foot bed, heel pad and memory foam foot pad.

2. The orthopedic appliance of claim 1 wherein said suede covering encloses the entire arch-support foot bed, heel pad, and memory foam foot pad.

3. The orthopedic appliance of claim 1 wherein said heel pad is made of an impact absorbing material with a density of 16-28 lbs/ft³, and a compression set percentage of under 10%.

4. The orthopedic appliance of claim 2 wherein said heel pad is made of an impact absorbing material with a density of 16-28 lbs/ft³, and a compression set percentage of under 10%.

5. The orthopedic appliance of claim 1 further comprising:
a forefoot pad made of a resilient shock absorbing material embedded on the bottom of the arch-support foot bed.

6. The orthopedic appliance of claim 2 further comprising:
a forefoot pad made of a resilient shock absorbing material embedded on the bottom of the arch-support foot bed.

7. The orthopedic appliance of claim 1 further comprising:
a lower heel pad made of a resilient shock absorbing material embedded on the bottom of the arch-support foot bed.

8. The orthopedic appliance of claim 2 further comprising:
a lower heel pad made of a resilient shock absorbing material embedded on the bottom of the arch-support foot bed.

9. The orthopedic appliance of claim 1 further comprising:
a shoe containing said orthopedic appliance wherein:
said shoe possesses a forefoot outsole pad made of a resilient shock absorbing material; and
said shoe possesses a heel outsole pad made of a resilient shock absorbing material.

10. The orthopedic appliance of claim 9 wherein the shoe has a separate forefoot outsole and a heel outsole.

11. The orthopedic appliance of claim 9 wherein the shoe has only one outsole.

12. The orthopedic appliance of claim 2 further comprising:
a shoe containing said orthopedic appliance wherein:
said shoe possesses a forefoot outsole pad made of a resilient shock absorbing material; and
said shoe possesses a heel outsole pad made of a resilient shock absorbing material.

13. The orthopedic appliance of claim 12 wherein the shoe has a separate forefoot outsole and a heel outsole.

14. The orthopedic appliance of claim 12 wherein the shoe has only one outsole.

15. An orthopedic appliance comprising:
a soft stretchy suede textile covering the top of a foot bed made of at least one material.

16. The orthopedic appliance of claim 15 wherein said material comprises anti-microbial, energy returning foam.

17. The orthopedic appliance of claim 15 wherein said material comprises an impact absorbing material.

18. The orthopedic appliance of claim 15 wherein said material comprises memory foam.

19. The orthopedic appliance of claim 15 wherein said material comprises a resilient shock absorbing material
